(19) United States
(12) Patent Application Publication
(10) Pub. No.: US 2007/0173949 A1
(43) Pub. Date: Jul. 26, 2007

(54) Bonding system for orthopedic implants

(76) Inventors: Lewis S. Sharps, Bryn Mawr, PA (US); Richard A. Balderston, Philadelphia, PA (US)

Correspondence Address: WERNER & AXENFELD, LLP, P. O. BOX 1629, WEST CHESTER, PA, 19380, US

(21) Appl. No.: 11/522,606

(22) Filed: Sep. 18, 2006

Related U.S. Application Data
(60) Provisional application No. 60/761,880, filed on Jan. 25, 2006.

(51) Int. Cl. A61F 2/28

(52) U.S. Cl. 623/23.5; 623/23.61

(57) ABSTRACT

A biocompatible bonding material is applied as an intermediary to attach prosthesis to boney tissue. The bonding material has the strength and rapid setting characteristics of PMMA cement, and is incorporated by the supporting bone as the boney ingrowth and osteogenic growth processes take place. In one embodiment, the bonding material is comprised of calcium phosphate polymer that provides immediate hardening and bonding, thereby locking the prosthesis to the bone. The biologic bond allows for bone in-growth over a period of weeks providing long term fixation. The immediate bonding can eliminate the early loosening that commonly occurred with uncemented joint replacements.

### CROSS-REFERENCE TO RELATED APPLICATIONS

The present patent application claims the benefit of U.S. Provisional Application Ser. No. 60/761,880 filed on Jan. 25, 2006. The content of the aforementioned application is fully incorporated by reference herein.

### TECHNICAL FIELD

This invention relates to prosthetic orthopedic implants, such as knees, hips, shoulders, ankles, discs, wrists, and other joint components. More specifically, this invention relates to a system and method of using a bonding material to create both immediate fixation between a prosthetic device and boney tissue and facilitate osteogenic growth of the boney tissue to the prosthetic material over time.

### BACKGROUND

The human body has a variety of movable orthopedic joints such as the knee joint, hip joint, shoulder joint, and the like. These joints are formed by the intersection of at least two bones. The intersecting end of each bone has smooth articular surface that is comprised of cartilage. As a result of injury, wear, arthritis, disease or other causes, it is occasionally necessary to replace all or part of an orthopedic joint with an artificial implant. This procedure is referred to as a joint replacement or arthroplasty. For example, a total knee arthroplasty comprises cutting off or resecting the articular surfaces at both the distal end of the femur and the proximal end of the tibia. Complementary artificial implants are then mounted on the distal end of the femur and the proximal end of the tibia. Where only a portion of a joint is damaged, a partial joint arthroplasty can be performed. In this procedure, one or more artificial implants replace only a portion of a joint.

Although joint replacement is now a common procedure, conventional implants and related mounting techniques have significant shortcomings. One significant drawback of many joint replacements is for example, the many existing materials used in conjunction with orthopedic implant repairs. That is, existing methods and materials present many unsatisfactory characteristics. Existing orthopedic implants are “walled off” by the body by a fibrous capsule as the result of the foreign-body protective response of the tissue in contact with the implant. This fibrous membrane or capsule may develop between the prosthesis, cement, and or bone thereby preventing a strong physical bond between the bone and the implant. Failure of a joint repair or replacement is often attributed to movement made possible by the presence of the soft fibrous capsule or membrane. The capsule gets progressively thicker as the implant ages in the body and the implant becomes more mobile and the motion exceeds a critical level.

Presently, the average service life of a prosthetic implant is about 12-15 years. About 50% of implants inserted into a younger population (less than age 40) need revision during the lifetime of the patient, subjecting the patient to additional surgery and the risks that accompany such procedures. The success rate is even lower for revision implants. Furthermore, a second revision is often fraught with increased risk of infection and or loosening of the prosthesis.

Failure of these systems is often the result of wear debris; particles of, polymethylmethacrylate (PMMA) cement and particles of metal often are separated from the prosthesis and invoke an inflammatory response, bone resorption, and pain, ultimately resulting in a loosening and failure of the entire prosthesis and or premature polyethylene wear. This usually occurs within the joint capsule. The tissue response includes granulation of tissue by a progressive foreign-body reaction, transforming the joint into a mass of reactive inflammatory fibrous tissue that can extend to the ligaments and muscles. Large areas of bone can become poorly vascularized and necrotic. The final stages of deterioration include resorption of the supporting bone.

The cement used to attach joint components to surrounding tissue is typically a PMMA cement, which may be modified by chemical additions for radio-opacity or short-term antibiotic activity. PMMA cements or hardens by an exothermic polymerization reaction. Full strength is obtained quickly, (usually within 10 minutes) so the cement has the advantage of providing support and fixation immediately after setting. The working time and setting time can be partially controlled to provide the surgeon with a surgically practical cement. It was the development of PMMA cement that made joint replacement possible.

Nevertheless, there are problems associated with its use. Cement is a brittle material with little resistance to the repeated loads experienced by joints. Furthermore, it has little adhesive properties. It acts simply as a grouting agent to fill the gaps between prosthesis and bone helping the bone to support the prosthesis. Loading and motion of the joint can produce fracture of the cement and separation of the cement from the prosthesis. Furthermore, rubbing between the prosthesis and cement can produce wear particles that are not well tolerated by the bone and produce local bone loss. Such loss makes the prosthesis loose and produces pain and loss of function and may require re-operation. Further, such bone loss greatly increases the difficulty of revision to a new prosthesis and greatly reduces the chance of a successful result.

For aged patients with short life expectancy the replacement of “broken hips” with a PMMA-cemented prosthesis was an improvement when it was first invented. For patients having longer lifetimes, there are serious problems as discussed below. The American Society for Testing and Materials specifies the following requirements (ASTM F-451) for PMMA cement:

| | |
|---|---|
| Working time | 5 minutes maximum |
| Setting time | 5–15 minutes |
| Strength | 70 MPa minimum |
| Solubility | 0.05 mg/cm³ maximum |
| Temperature rise | 90° C. maximum |
| Intrusion | 2.0 mm minimum |

The solubility is limited to reduce both local tissue and systemic responses (e.g., when the monomer is distributed systemically it can lower blood pressure and affect organs.). The temperature rise is limited to reduce the cauterization and death of tissue overheated by the exothermic setting reaction. The hazards associated with solubility and temperature rise are well recognized.

The cement must fill the space between the prosthesis component and the bone. The geometry of the prosthesis component is shaped to aid the load-bearing requirement. The prosthesis-to-PMMA bond and the PMMA-to-tissue bond participate in this. The prosthesis-to-PMMA bond is controlled by the bond chemistry and prosthesis geometry. The PMMA-to-tissue bond is controlled by the tissue reactions and the body's physiological response. Initially this response includes bone resorption and then reconstruction through bone healing mechanisms to repair the damage produced by the surgical trauma and the temperature rise. When first inserted, the PMMA is smooth and undesirable tissue response is limited. With time, the PMMA cement can fatigue and become subject to fragmentation, and cracking releasing PMMA debris into the joint. The fragments of cement invoke an inflammatory response in the surrounding tissue and the cracks provide fresh surfaces for chemical exchange. The PMMA is weakened and subject to movement. Inflammation and tissue resorption further weakens the PMMA-to-tissue interface, ultimately, resulting in failure of the prosthetic device. The most common patient complaint associated with prosthesis failure and device loosening under stress (at one of the two bond sites) is progressive pain.

Another associated problem is that there is no physiologic bond or healing between the PMMA and the bone tissue. Instead a mechanical bond is achieved by forcing the fluid PMMA cement, under pressure, into the bone to penetrate pores and irregularities in the bone geometry. Sometimes a dam is inserted in the intramedullary space to restrict the longitudinal flow of the PMMA cement and obtain higher pressure and more radial flow. As an example, the subcortical region is an important load-bearing area composed of trabecular bone with the trabeculae oriented to transmit the load from one load-bearing region to another. The trabeculae are strong, thin regions of bone, forming the mesh-like interiors of spongy bone, commonly growing along stress lines. Their blood supply comes from the pores (also oriented by the trabeculae orientation) and from the intramedullary region, from attached tendons and from surrounding muscle, although the latter is usually less important. When a blood supply is removed by surgery, it must be compensated by other sources. This is not possible if the pores supplying blood are blocked by the PMMA cement.

Thus, inherent in the use of PMMA cement is an undesirable interference with blood supply. Although PMMA cements contribute immediate strength to the bone by filling the pores and supporting the trabeculae, such cements do not have enough strength when the trabeculae become seriously weakened, which is all but inevitable. Therefore the use of PMMA cement presents a basic limitation to the longevity of an implant. The cement breakdown and the PMMA-induced tissue response can prevent implants from lasting throughout extended life spans of patients. For these reasons a ten to fifteen year life is probably the maximum to be expected.

Another limitation of PMMA cement is the lack of bonding between metal and PMMA cement. Present practice usually provides a modified prosthetic undersurface to obtain mechanical interlocking between the cement and the prosthesis to attempt to compensate for this deficiency. Prosthetic devices used for cemented joint replacement are made of strong materials such as metal, cobalt-chromium or titanium, the surfaces of which are smooth and non-porous. While the use of such materials allows the new joint to withstand the stress of load-bearing, the smooth surface impedes bone ingrowth. As the prosthetic device ages and it becomes necessary to remove or replace the implant, it is difficult to remove the non-porous PMMA-bonded implant without fracture or damage to healthy bone. If PMMA cement is used with a prosthetic device having a porous surface, the problem of bone fracture and breakage upon removal or replacement is even more severe. Since most joint replacements will at some point require replacement, the issue of further damage to healthy bone is a serious concern for orthopedic surgeons.

The use of porous implants is another technique, which involves fixation of metallic prostheses to bone without the use of cement. This cement-less technique avoids the problems associated with cement but introduces its own problems. Cement fracture and its effects are eliminated. Metallic wear debris is reduced but is still present and can result in damage to bone and adjacent soft tissue. The most important new problem introduced by cement-less implants using porous prostheses is poor initial fixation. Cement provides instant, excellent, initial fixation. This fixation may degrade with time and use but it is usually excellent initially. Fixation of a porous coated device initially relies on a press fit, which may be difficult to achieve. Further, there is no initial impregnation of the fixation means into the bone and thus, such press fit is inferior to cement in attaching the prosthesis to bone. Biological ingrowth, or impregnation, relies on a stable connection between prosthesis and bone. If relative motion is not essentially eliminated, ingrowth will not occur, a fibrous capsule or membrane will develop and biological fixation will not be achieved.

In cases with cemented porous implants or implants with pre-coating to bond the implant to the cement, removal of the components again may not be easy. In these situations, it is often difficult to determine the plane between hard bone and hard cement, causing operative difficulties.

An important consideration in orthopedic surgery is the ability of bone to bond to the implant. It is well-known that hydroxyapatite and calcium phosphate are biocompatible and can provide a scaffold to allow bony ingrowth. This has led those in the field to investigate bone cements in which hydroxyapatite (HA) or modifications of HA are used to form a cement-like agent. Commercial cements are available based on precipitated HA or modified HA. However, in this application, bone substitute materials are used primarily to “fill space” or help in stabilizing bone, and can also in some situations be used as load-bearing members.

### SUMMARY

As described above in the Background section, the concept of biologic bonding has been developed utilizing HA or hydroxyapatite spray. HA is sprayed onto the prosthesis during its manufacture and bonds onto the bone within 2-6 weeks eliminating the fibrous layer between cement and bone. This, however, does not provide any immediate fixation between prosthesis and bone. Immediate fixation still relies upon a press fit construct.

To overcome this and other problems inherent with current practices in the art, described herein is a new generation of bonded joint prosthesis systems that provide for immediate fixation and long term bone (osteogenic) ingrowth. That is, the following discussion introduces the broad concept of a using a biocompatible bonding material (not PMMA cement) capable of immediate fixation and more physiologic transmission of load between boney tissue and an implant, in conjunction with an orthopedic implant containing a porous surface, for joint replacement surgery. The bonding material will have the strength and rapid setting characteristics of PMMA cement, and will be incorporated by the supporting bone as the boney ingrowth and osteogenic growth processes take place. Accordingly, the bonding material and associated bonding prosthesis have the ability to adhere and conform to the implanted site and facilitate bone growth, to deter ingrowth of non-bone tissue into the implant site, to be immunologically tolerated by the host, and to serve as a framework for the newly forming bone tissue.

In one embodiment, the bonding material is comprised of calcium phosphate polymer that provides immediate hardening and biological bonding when interfacing an orthopedic implant to a boney tissue, thereby locking the implant to the bone. The biologic bond will also allow for bone in-growth over a period of 6-10 weeks providing long term fixation. The immediate bonding can eliminate the early loosening that commonly occurred with traditional uncemented total knee replacements.

The physical property of the surface of the implant which abuts and is bonded to boney tissue is preferably comprised of a metal material such as titanium or cobalt chrome. A portion of the implant surface has a textured, trabeculated or porous surface. In one embodiment, the optimum pore size of the porous surface is between 50 μm to 400 μm. The pore size may be spread over the load-bearing sections of the implant surface with the texture mirroring that of trabeculae boney tissue. Thus, the porous implant surface interfaces with the synthetic calcium phosphate polymer, and in turn is bonded to the surface of boney tissue thereby providing an artificial joint system.

Various embodiments of the present invention provide an orthopedic bonding system for artificial implants in which an artificial joint component with a porous surface is bonded to the surface of boney tissue by a biocompatible non-cement bonding material. Such a bonding material provides immediate strength and rapid setting characteristics of PMMA cement, as well as long term bone in-growth fixation between the prosthetic device and adjacent bone.

### BRIEF DESCRIPTION OF THE DRAWINGS

The detailed description is presented with reference to the accompanying figures. In the figures, the left-most digit(s) of a reference number identifies the figure in which the reference number first appears.

FIG. 1 shows a bonded artificial joint system.

FIG. 2 shows a bonded knee joint system.

FIG. 3 shows a bonded hip joint system.

FIG. 4 shows a bonded shoulder joint system.

### DETAILED DESCRIPTION

The following description details the concept of a using a biocompatible bonding material (not PMMA cement) in conjunction with an orthopedic implant containing a porous surface, whereby the porous implant can be bonded to a boney tissue, providing a superior method of orthopedic joint replacement overcoming many of the shortcomings of cement and cement-less joint replacement procedures currently performed. The biocompatible bonding material provides for immediate fixation of the implant to boney tissue; the bonding material will have the strength and rapid setting characteristics of PMMA cement. The bonding material of the present invention also provides immediate fixation and load bearing properties between implant and boney tissue, while promoting boney ingrowth. Accordingly, the bonding material and associated bonding prosthesis have the ability to rapidly adhere to and conform to the implanted site and facilitate bone growth, to deter ingrowth of non-bone tissue into the implant site, to be immunologically tolerated by the host, and to serve as a framework for the newly forming bone tissue.

The biocompatible non-cement bonding material and corresponding orthopedic implant configured with a porous surface may be recommended to medical personnel for use as an artificial joint system in orthopedic surgery. Such a system may include materials packaged together or materials that are available and packaged separately. In either event, materials can be sold with information and potentially training as to how they would be used together in surgery when performing partial or full joint replacement procedures. In one embodiment, the system includes an artificial joint component comprising a porous surface on at least a portion of the surface of the artificial joint component. The porous surface is for interface with a boney tissue. A non-cement biocompatible bonding material provides for immediate fixation of the porous surface of the artificial joint component to the boney tissue in situ. Components of the bonded artificial joint system are typically packaged aseptically so that they are suitable for use in surgery when removed from packaging.

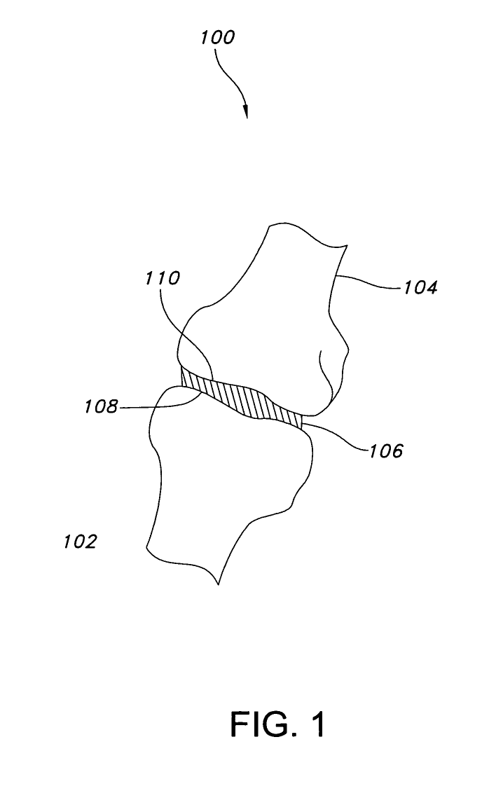

Referring to FIG. 1, there is shown a bonded artificial joint system 100, including an artificial joint component 102, a boney tissue 104, and a bonding material 106. The bonding material 106 bonds the artificial joint component 102 to the boney tissue 104 by interfacing a surface 108 of the artificial component with a surface 110 of the boney tissue.

The artificial joint component 102 is any suitable prosthetic device or material used for implantation in orthopedic surgery and is comprised of various materials such as chrome, titanium, ceramic, rubber and plastic, and any combination of such. As used herein, a prosthetic device is any artificial component used in joint replacement surgery, in which an orthopedic joint is either partially or fully replaced, such as but not limited to; knee replacement surgery, hip replacement surgery, shoulder replacement surgery, or any other joint replacement surgery involving replacement of joint tissue with an artificial component.

The physical property of the surface of artificial joint component 102 which abuts boney tissue 104 may be comprised of a metal material such as titanium, cobalt chrome, ceramic material or any combination of such materials or other materials suitable for use as part of the surface of an artificial joint component. The surface 108 of artificial joint component 102 is textured, trabeculated or porous. In one embodiment, the optimum pore size of the porous surface is between 50 μm to 400 μm. The pore size may be spread over the load-bearing sections of the implant surface with the texture mirroring that of trabeculae boney tissue.

Boney tissue 104 is a non-artificial component to which an artificial component is attached, and is part of a natural human joint system, such as the femur, tibia and patella of the knee, or any other boney tissue of a joint system, such as the hip, shoulder or spine. Boney tissue 104 is generally mechanically reshaped to provide the surface 110 for accepting, and bonding to, artificial joint component 102 upon the addition of bonding material.

Bonding material 106 is any suitable biocompatible agent that is strong or stronger than necessary to provide suitable orthopedic reinforcement for prosthetic implants while promoting osteogenic growth at the site of implantation. Bonding material 106 may be applied at the site of joint replacement, i.e., to the boney tissue prior to the insertion of artificial joint component 102 at the time of surgery, or bonding material 106 may be applied directly upon the porous surface 108 of artificial joint component 102 prior to the surgical procedure involving joint replacement.

In one embodiment, bonding material 106 is comprised of a calcium phosphate polymer that provides rapid, if not immediate, hardening and bonding thereby locking the artificial joint component 102 to boney tissue 104. Calcium phosphate polymer is comprised of nonimmunogenic beta-tricalcium phosphate of nano-sized particles that enhances biologic bone ingrowth through a simultaneous process of Calcium phosphate boney incorporation. In this manner there is no loss of initial fixation during the boney ingrowth process and new bone growth. Calcium phosphate polymer integrates into existing bone, facilitating new bone formation in six weeks. The porosity and interconnected structure of the calcium phosphate polymers provides a scaffold for new bone ingrowth, vascularization, and osteoconduction. Suitable calcium phosphate polymer is available under the product name Vitoss® and can be purchased from Orthovita®, a company located at 45 Great Valley Parkway, Malvern, Pa. 19355.

In another embodiment, bonding material 106 is a terpolymer resin with combeite glass-ceramic reinforcing particles that provide immediate bonding to bone and improved mechanical strength of the prosthetic implant. One suitable glass-ceramic resin is comprised of combeite glass-ceramic particles; barium boro-aluminosilicate glass and amorphous silica, bound in a terpolymer resin comprised of bisphenol-a-glycidyl dimethacrylate, bisphenol-a-ethoxy dimethacrylate, and triethylene glycol dimethacrylate. Glass-ceramic bonding material provides biocompatible tissue interface, mechanical strength, direct bone apposition and bonding, and rapid setting for immediate load. Suitable glass-ceramic particle resin is available under the product name Cortoss® and can be purchased from Orthovita®, a company located at 45 Great Valley Parkway, Malvern, Pa. 19355.

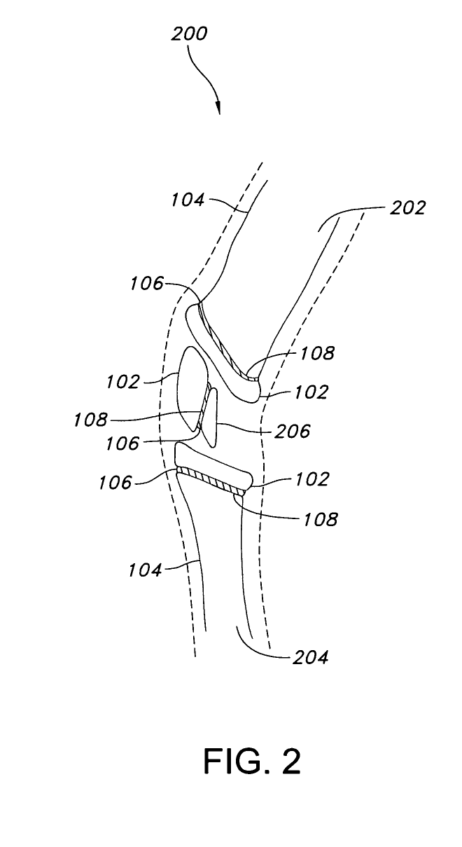

Referring to FIG. 2, there is shown an artificial knee joint arrangement 200. In one implementation, artificial joint component 102 is a prosthetic device suitable for implantation to restructure an area of at least one of the femur 202, tibia 204 or patella 206. Bonding material 106 is interfaced between the surface 108 of the artificial joint component 102 and the boney tissue 104.

The artificial knee joint 200 is immediately capable of load bearing and will undergo boney ingrowth, with new bone growth appearing in as early as six weeks. The method is suitable for partial or total knee replacements.

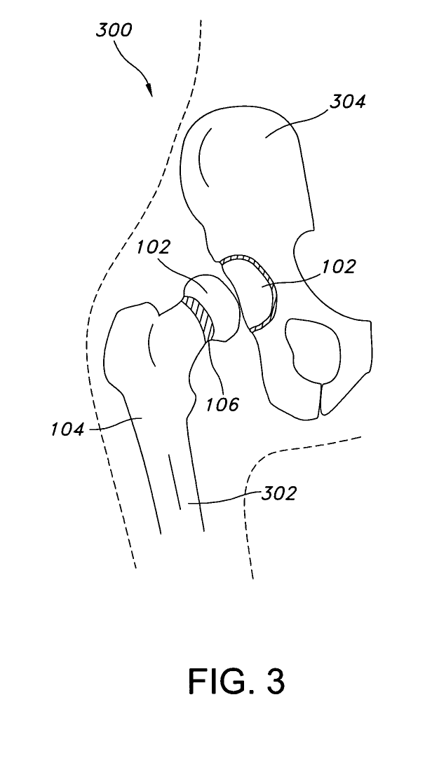

FIG. 3 shows a bonded hip joint prosthesis 300. Boney tissue 104 is femur 302 and hip socket 304, damaged portions of which are removed and replaced by bonding artificial joint component 102 to boney tissue 104 by bonding material 106. The method is suitable for partial or total hip replacements.

In one embodiment, artificial joint component 102 is a metal ball and stem and is fixed to boney tissue of femur 302 by bonding material 106.

In another embodiment artificial joint component 102 is a plastic or metal cup and is fixed to boney tissue 104 of hip socket 304.

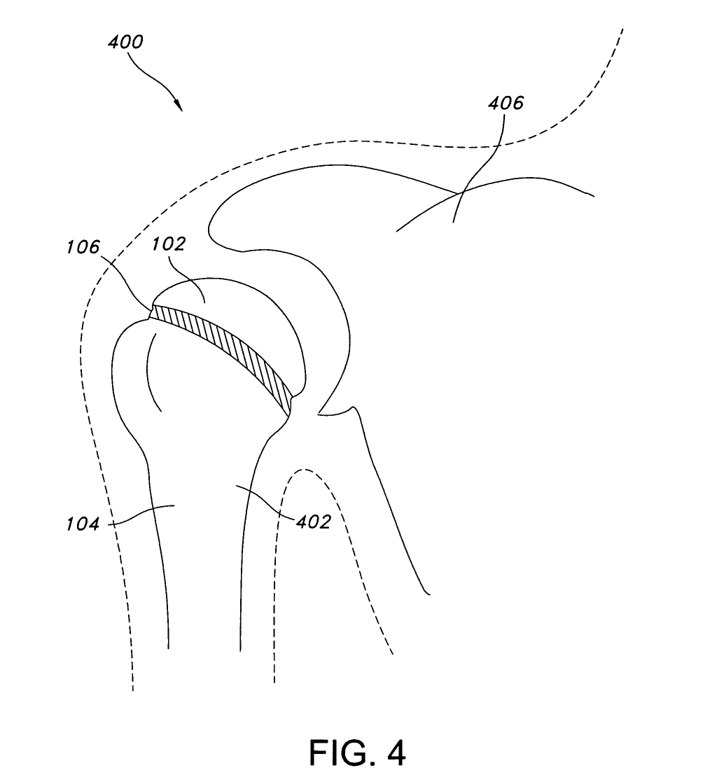

FIG. 4 shows a bonded shoulder joint prostheses 400. Boney tissue 104 of the shoulder joint includes humerus 402 and scapula 406, damaged portions of which can be removed and replaced by bonding artificial joint component 102 to boney tissue 104 by bonding material 106. The method is suitable for partial replacements and resurfacing of the shoulder joint.

Reference herein to “one embodiment”, “an embodiment”, “an implementation” or “one implementation” or similar formulations herein, means that a particular feature, structure, operation, or characteristic described in connection with the embodiment, is included in at least one embodiment of the present invention. Thus, the appearances of such phrases or formulations herein are not necessarily all referring to the same embodiment. Furthermore, various particular features, structures, operations, or characteristics may be combined in any suitable manner in one or more embodiments.

In the foregoing description, for purposes of explanation, specific numbers, materials and configurations are set forth in order to provide a thorough understanding of the present invention. However, it will be apparent to one skilled in the art that the present invention may be practiced without each specific example. In other instances, well-known features are omitted or simplified to clarify the description of the exemplary embodiments of the present invention, and thereby, to better explain the present invention.

The inventors intend these embodiments and implementations to serve as representative illustrations and examples. The inventors do not intend these embodiments to limit the scope of the claims; rather, the inventors have contemplated that the claimed invention might also be embodied and implemented in other ways, in conjunction with other present or future technologies. Thus, the embodiments described herein are to be considered in all respects only as exemplary and not restrictive.

What is claimed is:

1. A method, comprising: using a biocompatible bonding material as an intermediary between an artificial joint component and a boney tissue to provide immediate fixation of the artificial joint component to the boney tissue and wherein the bonding material promotes osteogenic growth between the artificial joint component and the boney tissue in situ and wherein the artificial joint component comprises a porous surface on at least an undersurface of the artificial joint component.

2. The method of claim 1, wherein the biocompatible binding material is a calcium phosphate polymer.

3. The method of claim 1, wherein the biocompatible bonding material is a glass-ceramic resin.

4. The method of claim 1, wherein the artificial joint component is a prosthetic device used for orthopedic implantation.

5. The artificial joint component of claim 1, wherein the pore size is between approximately 50 μm and 400 μm.

6. The method of claim 1, wherein the boney tissue is a non-artificial component of a natural human joint system.

7. A bonded artificial joint system for use in surgery, comprising:
an artificial joint component comprising a porous surface on at least a portion of the surface of the artificial joint component for interface with a boney tissue; and
a non-cement biocompatible bonding material for providing immediate fixation between the porous surface of the artificial joint component and the boney tissue in situ.

8. The bonded artificial joint system of claim 7, wherein the artificial joint component is a prosthetic device used for orthopedic implantation.

9. The artificial component of claim 7, wherein the porous surface has a pore size of between approximately 50 μm and 400 μm.

10. The artificial joint system of claim 7, wherein the boney tissue is a non-artificial component of a natural human joint system.

11. The artificial joint system of claim 7, wherein the non-cement biocompatible bonding material is a synthetic calcium phosphate polymer.

12. The artificial joint system of claim 7, wherein the non-cement biocompatible bonding material is a glass-ceramic resin.

13. A method of joint replacement, comprising:
disposing a biocompatible bonding material on a porous surface of an artificial joint component; and
interfacing the surface of the artificial joint component having the biocompatible bonding material disposed thereon to the surface of the boney tissue.

14. The method of claim 13, wherein the artificial component is a prosthetic device used for orthopedic implantation.

15. The method of claim 13, wherein the pore size is between approximately 50 μm and 400 μm.

16. The method of claim 13, wherein the bonding material is a non-cement biocompatible bonding material capable of providing fixation of the artificial joint component to the boney tissue, and wherein the bonding material promotes osteogenic growth of the boney tissue at the sight of fixation.

17. The method of claim 13, wherein the bonding material is a calcium phosphate polymer.

18. The method of claim 13, wherein the bonding material is a glass-ceramic resin.
